(19) United States
(12) Patent Application Publication
(10) Pub. No.: US 2026/0241110 A1
(43) Pub. Date: Aug. 20, 2026

(54) Medicament delivery device and medicament delivery assembly

(71) Applicant: SHL Medical AG, Zug (CH)

(72) Inventors: Yun Hsuan Chang, Taoyuan City (TW); Tzu-Chia Lee, Taoyuan City (TW); Chiao-Yi Chiu, Taoyuan City (TW)

(21) Appl. No.: 19/160,554

(22) PCT Filed: Feb. 28, 2024
(86) PCT No.: PCT/EP2024/055097

§ 371 Date: Aug. 28, 2025

(30) Foreign Application Priority Data
Mar. 13, 2023 (EP) 23161504.8

(51) Int. Cl. A61M 5/315; A61M 5/24

(52) U.S. Cl. CPC A61M 5/3157; A61M 5/2422; A61M 5/31583; A61M 2205/581

(57) ABSTRACT

A medicament delivery device for accommodating a medicament container is disclosed. The medicament delivery device comprises a base unit with a housing extending along a longitudinal axis and a drive unit arranged to be subject to an expelling movement relative to the base unit into a proximal direction, to act on the medicament container for expelling a medicament therefrom. One of the drive unit and of the base unit further comprises a revolving member and the other one comprises a cooperating structure, wherein the revolving member (42) is mounted to be caused to rotate upon the expelling movement of the drive unit. One of the revolving member and the cooperating structure comprises a striker and the other one comprises a control surface with a plurality of teeth. When the revolving member rotates, the striker moving along the control surface and moving over the teeth causes audible signals.

### TECHNICAL FIELD

The invention is in the field of medicament delivery devices. More in particular, it relates to an automatic medicament delivery device providing audible feedback to a patient who uses it. It further relates to an assembly comprising such a device, pre-assembled with a medicament container.

### BACKGROUND

Medicament delivery devices for automatic delivery of a medicament by self-administration are well-known. Especially, they may be equipped to accommodate a medicament container, for example a medicament container with a septum to be perforated immediately prior to use, or a syringe. Often, the medicament delivery device and the medicament container are pre-assembled to constitute a medicament delivery assembly for self-administration.

Such medicament delivery devices and medicament delivery assemblies should generally be safe to use and easy to handle, and they should ensure delivery of the designated dose of the medicament. In view of this, it may be important that the patient receives a feedback signal during and/or after delivery of the medicament.

U.S. Pat. No. 7,758,550 and WO 2017/129337 disclose medicament delivery devices in which a plunger rod moves, for delivering the medicament, urged by a biasing member, in proximal direction. This movement of the plunger rod causes latching elements or bumps to interact with an engaging structure moved relative thereto in an axial direction, so that discrete acoustic ‘click’ signals are generated during medicament delivery. The distances between adjacent latching elements or bumps may vary so that when the speed of the plunger rod diminishes towards the end of the delivery process, the time interval between the discrete acoustic signals remains constant. This approach, however, has its limits in terms of flexibility. Also, it requires that the engaging structure, for example a flex arm, is subject to a relatively small resilient force so as to not impede the movement of the plunger rod. As a consequence, the engaging structure, being a part of the plunger rod, needs to have a relatively weak material or delicate structure.

### SUMMARY

Accordingly, it is an object of the present invention to provide a medicament delivery device that overcomes at least some of the drawbacks of the prior art. Especially, the medicament delivery device should be capable of providing reliable feedback signals during delivery without compromising the stability and reliability of the parts that

This object is achieved by the invention as defined in the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

When a component is said to move proximally, distally, axially in a proximal direction, axially in a distal direction, in a circumferential direction, or equivalent terms, the movement is relative to the housing of the injection device, unless mentioned otherwise.

According to an aspect of the present invention, a medicament delivery device for accommodating a medicament container is provided. The medicament delivery device comprises a base unit with a housing extending along a longitudinal axis and a drive unit arranged to be subject to an expelling movement relative to the base unit into a proximal direction, to act on the medicament container for expelling a medicament therefrom. If the medicament container has a plunger or stopper, the drive unit may for example act, by being moved into the proximal direction, on the plunger or stopper to expel the medicament via a needle, which needle may belong to the medicament delivery device, or, for example if the medicament container is a syringe, to the medicament container.

One of the drive unit and of the base unit further comprises a revolving member, and the other one comprises a cooperating structure. The revolving member is mounted to be caused to rotate, especially about the longitudinal axis, upon the expelling movement of the drive unit. One of the revolving member and the cooperating structure comprises a striker, and the other one comprises a control surface with a plurality of teeth, wherein, when the revolving member rotates, the striker moving along the control surface and moving over the teeth causes audible signals.

Especially, the striker may be resilient and may abut against the control surface and be constantly in physical contact with it. When the striker moves/slips over the teeth due to the rotation of the revolving member, it is further deflected resiliently. When it gets out of engagement with a tooth, it jumps back, thereby hitting the control surface next to the respective tooth, thereby generating an acoustic signal.

The teeth can have any shape suitable for the purpose of causing the striker to generate the signals when being moved over them. Especially, in embodiments, the teeth may form a ramp along which the striker is lifted against an elastic force when moving over the tooth, followed by a radial (‘vertical) or undercut (‘overhanging’) face allowing the striker to jump back and to impinge on the control surface for causing a click-like acoustic signal.

For the revolving member being caused to rotate by the expelling movement the medicament delivery device may comprise a helical feature and an engaging feature engaging with the helical feature. The helical feature may for example be a helically running groove, with the engaging feature being an engaging protrusion engaging into the helically running groove. Alternatively, the helical feature may be a helical protrusion, and the engaging feature may be an engaging notch or similar into which the helical protrusion engages. The helical feature and the engaging feature may also be an outer and an inner thread or inner and outer thread, respectively.

In this, one of the helical feature and of the engaging feature belongs to the revolving member, and the other one belongs to the assembly other than the one to which the revolving member belongs (for example to the drive unit if the revolving member belongs to the base unit). Thereby, the expelling movement includes a relative axial movement of the helical feature and of the engaging feature, whereby the expelling movement causes the revolving member to rotate.

The helical feature may for example be a helically running groove or ridge extending along a shaft or tube of the revolving member, and the engaging feature may belong to the drive unit, for example to a guide tube or guide shaft, respectively, thereof.

The inclination of the helical feature—the helix angle—can be constant along its course. Alternatively, the inclination may be non-constant. Especially, the inclination may vary in addition or as an alternative to a variation of the distance between the teeth varying (see below) for compensating for variations in the speed of the drive unit.

Especially, both, the revolving member and the cooperating structure may belong to the base unit, whereby the revolving member rotates, during the expelling movement, relative to the housing but does not move axially. The cooperating structure may then be stationary relative to the housing, for example by being a structure of a cap or similar affixed to the housing, or by being a structure of the housing itself.

Especially, the revolving member may comprise the striker being a ‘clicker’ extension, especially of a distal head of the revolving member, capable of being deflected resiliently by the teeth.

For example, the striker may be an outward extension of such a distal head of the revolving member. The control surface may then be an inner surface of the cooperating structure, the striker being deflected towards radially inwardly by abutting against the control surface. The revolving member may further comprise the mentioned shaft, extending towards proximally from the distal head.

The housing of the medicament delivery device may comprise a housing sleeve, thus a tubular portion, for example with a distal end plate. In addition, it may comprise a distal end cap member. In embodiments, the distal end cap member in addition to forming a distal end cap of the housing also comprises the cooperating structure, especially comprising the control surface. For example, the control surface may be an inner annular surface of a shallow proximal indentation of the distal end cap member.

In embodiments with the housing comprising a housing sleeve, the housing sleeve has a distal end plate with an end plate hole, wherein a distal head of the revolving member is arranged distally of the distal end plate, and the shaft extends through the end plate hole. The distal end plate fixes an axial position of the revolving member and prevents it from moving towards proximally.

In addition to comprising a housing sleeve—with or without the distal end plate—and/or a distal end cap member, the housing may comprise a container housing accommodated within the sleeve and providing a seat for the medicament container.

An advantage of the approach according to the present invention is that, in contrast to the prior art, the revolving member, being the part the movement of which causes the signals, is a part separate from the drive unit. It may thus have a delicate structure without compromising the stability of the drive unit. Also, the designs of the striker and of the cooperating structure may be chosen essentially freely, due to the revolving member being a separate part.

In embodiments the teeth have unequal distances to each other. Especially, a variation of the distances between the teeth may compensate for variations in the speed of the expelling movement, so that the time intervals between subsequent signals are approximately equal.

The drive unit may comprise a plunger rod that acts on a plunger or stopper of the medicament container by being moved towards proximally in the expelling movement. By pushing the plunger or stopper towards proximally, it expels the medicament.

For causing the expelling movement, the medicament delivery device may comprise a biasing member equipped to move the drive unit towards distally during the expelling movement. The biasing member may for example be a pre-tensioned drive spring.

In embodiments, such drive spring may be at least partially accommodated in an interior of the plunger rod and on one side abut against an element of the base unit (for example a distal end plate of the housing, for example a housing sleeve thereof) to urge the plunger rod towards distally as soon as the drive unit is released.

Activation of the drive unit by releasing it may, as is known from the prior art, be automatic. For example, the medicament delivery device may comprise a needle guard protecting a needle (of the medicament delivery device or of the medicament container, the needle being for injecting the expelled medicament under the patient's skin) after a proximal end cap or similar has been removed, immediately prior to use. The automatic release may be caused by the needle guard being displaced, against a biasing force, towards distally when the patient presses the medicament delivery assembly against the dose delivery site. To this end, the medicament delivery device may comprise a drive locking member that prevents the drive unit from moving towards proximally prior to release. Release may for example be due to a rotation of the drive locking member, such rotation caused by the displacement of the needle guard towards distally.

In addition to concerning a medicament delivery device, the present invention also concerns a medicament delivery assembly comprising the medicament delivery device of the kind described in the present text, pre-assembled with a medicament container, for example a medicament container with a septum to be pierced by a needle of the medicament delivery device prior to use, or a syringe with a pre-assembled needle.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings. The drawings show:

FIG. 1 a view of an embodiment of a medicament delivery assembly comprising the medicament delivery device, the medicament delivery assembly already prepared for medicament delivery;

FIG. 2 an exploded view of the medicament delivery device, with some components assembled with each other;

FIG. 3 the medicament container;

FIG. 4 the plunger rod of the medicament delivery device of FIGS. 1 and 2, together with the drive spring, the guide rod, the revolving member and the distal end cap member, cut along an axially extending plane;

FIG. 5 a distal end portion of the housing sleeve;

FIG. 6 the elements of FIGS. 4 and 5 assembled together;

FIG. 7 a view of the elements shown in FIG. 4 together with the medicament container during medicament delivery, and the housing sleeve;

FIG. 8 the guide tube and the revolving member;

FIG. 9 the distal end cap member; and

FIG. 10 the plunger rod and the guide tube.

### DETAILED DESCRIPTION

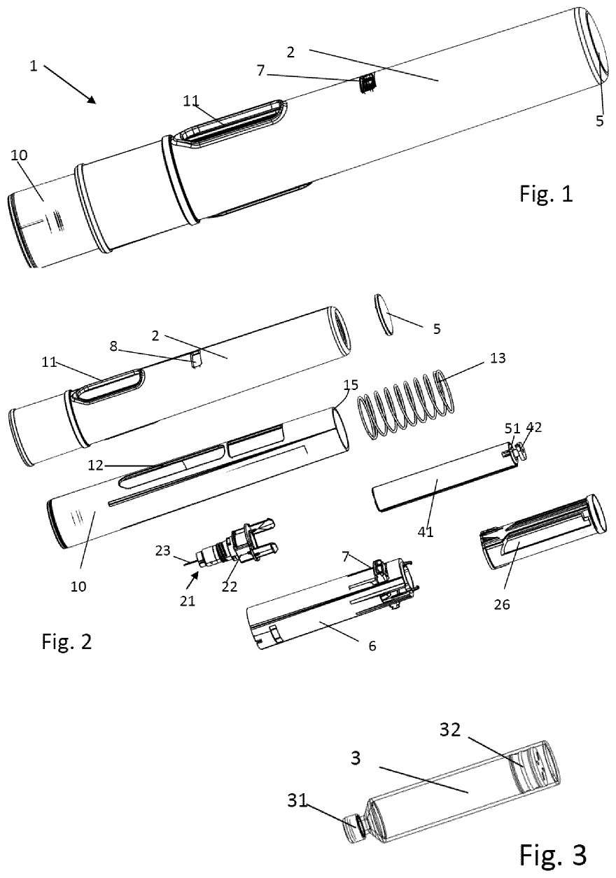

The medicament delivery assembly 1 shown in FIG. 1 comprises a medicament delivery device and a medicament container 3. FIG. 2 shows an exploded view of the medicament delivery device, and FIG. 3 depicts the medicament container 3 being a medicament container (or ‘cartridge) with a proximal septum to be pierced prior to use. The medicament container 3 is shown transparent, with a head portion 31, and with a stopper 32 on the inside.

The medicament delivery device comprises a housing, encasing, possibly together with a removable proximal end cap (not shown in the figures, removed prior to use), the medicament container 3. In the depicted embodiment, the housing comprises a housing sleeve 2 and a distal end cap member 5 and further comprises an—optional—container housing 6 in which the container is seated. The container housing 6 is fixedly connected to the housing sleeve 2 by way of fastening protrusions 7 engaging into fastening cut-outs 8 of the housing sleeve 2.

The medicament delivery device further comprises an activation member, namely a needle guard 10. The needle guard is arranged at least partially inside of the housing sleeve 2 and around the container housing 6. It has a portion that, in the ready-for-delivery configuration of FIG. 1, proximally extends out of the housing sleeve 2 to protect the needle.

The container housing 6 may at least partially be made of a transparent material, and the medicament container 3 may be partially visible through a housing window 11 and a needle guard window 12, whereby the patient can inspect the content of the medicament container 3 if the medicament container is made of transparent material, also.

Distally of the needle guard 10, the medicament delivery device comprises a needle guard spring 13 accommodated in a spring compartment 14 (see FIG. 5) within the housing sleeve, between a distal end face 15 of the needle guard 10 and a distal end plate 16 of the housing sleeve 2.

The medicament delivery device further comprises a needle assembly 21 that has a distal retainer fixedly connected to the container housing 6 and shaped to accommodate a head portion 31 of the medicament container. The needle assembly 21 is equipped for a needle sub-assembly comprising the needle 23 to be moved towards distally so as to pierce the septum at the head portion 31 of the medicament container 3 in an initial preparation process. A possible mechanism for this is for example described in WO 2013/089616. In FIG. 2, the needle assembly 21 is shown in a configuration after this initial preparation step, which is for example caused by a removal of the proximal end cap (not shown in the figures) carried out to bring the medicament delivery device into the state shown in FIG. 1.

As an alternative to comprising a needle assembly, for example as shown in FIG. 2, the medicament delivery device could also be configured to accommodate a syringe as the medicament container, in which case the needle is pre-assembled with, and belongs to, the medicament container. Generally, the working principle of the present invention neither depends on the nature of the medicament container nor on the needle but is applicable to any configuration in which the medicament is expelled by moving a drive unit, for example comprising a plunger rod, into an axial, especially proximal, direction.

The medicament delivery device further comprises a drive locking member 26 (sometimes called ‘rotator’) and a plunger rod 41, together with further elements described in more detail hereinafter. The plunger rod 41, if applicable together with further parts moving with it, forms the drive unit.

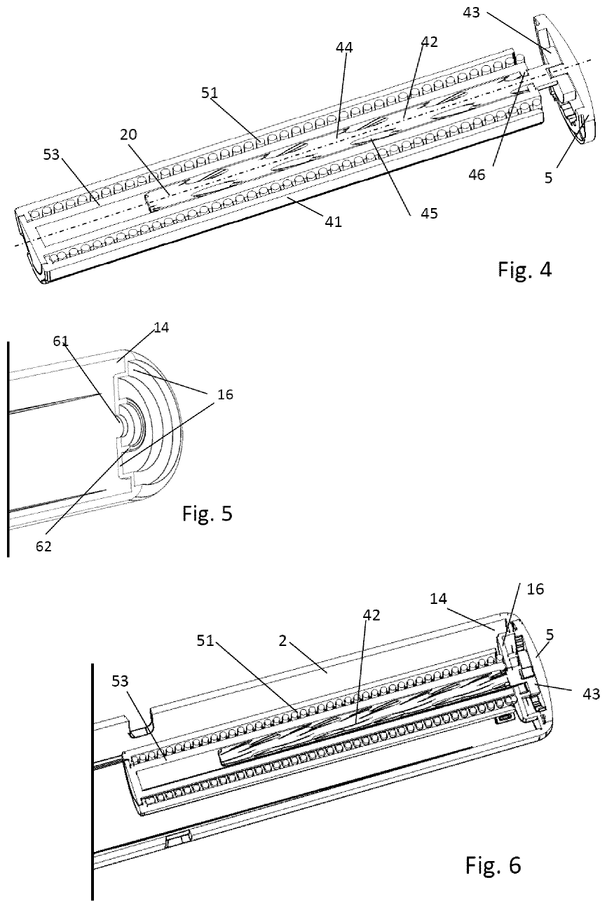

For activation, the patient presses the medicament delivery assembly of FIG. 1, thus with the proximal end cap removed, against the dose delivery site and thereby displaces the needle guard 10 towards distally relative to the housing, against a spring force of the needle guard spring 13. As a result, the needle 23 becomes exposed and can penetrate the patient's skin at the dose delivery site. At the same time, the needle guard 10 interacts with the drive locking member 26 to rotate the same about the longitudinal axis 20 (see FIG. 4), by a certain angle. The drive locking member 26 will, by this movement, release the drive unit and allow it to move axially into the proximal direction (expelling movement) so that the plunger rod 41 acts on the medicament container's stopper (or plunger). Thereby, it activates the medicament delivery.

There are several possible mechanisms by which a movement of an activation member—such as the needle guard 10—relative to the housing can activate the medicament delivery. Since the present invention does not depend on the nature of this activation, it is not described in any more detail here.

FIGS. 4-10 illustrate how signals are generated during medicament delivery due to the axial movement of the drive unit.

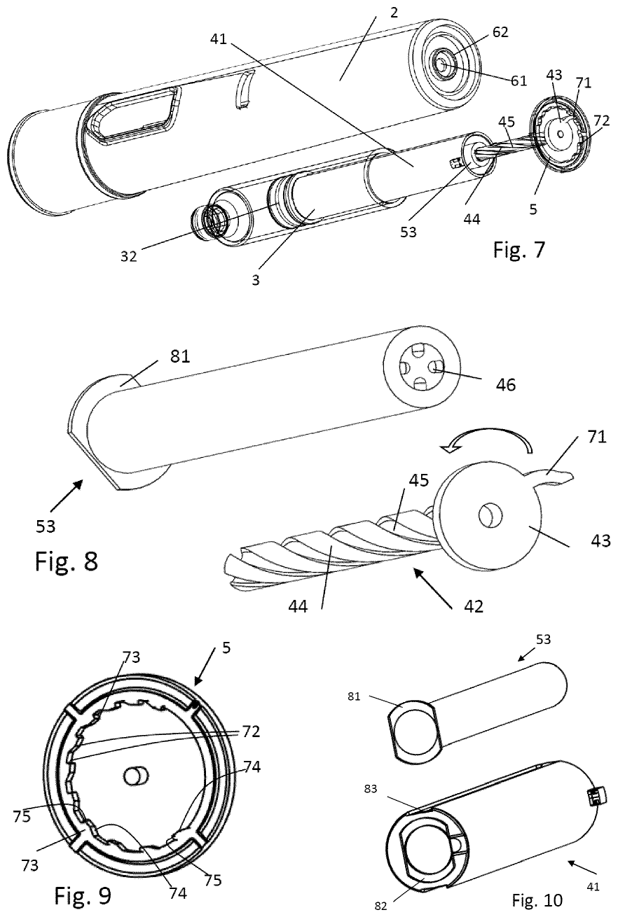

FIG. 4 shows the plunger rod 41 together with the drive spring 51, the guide rod, the revolving member 42 and the distal end cap member 5, cut along an axially extending plane. FIG. 5 depicts a distal end portion of the housing sleeve, and FIG. 6 shows the elements of FIGS. 4 and 5 assembled together (but, for ensuring a clearer view, without the needle guard spring, the drive locking member and the distal portion of the needle guard that in the assembled state of the medicament delivery device extend around the plunger rod 41, and also without the other components of the medicament delivery assembly). FIG. 7 depicts a view of the elements shown in FIG. 4 (with the distal end cap member 5 shown transparent) together with the medicament container 3 during medicament delivery as well as the housing sleeve. FIG. 8 shows enlarged views of the guide tube and of the revolving member, FIG. 9 shows an even more enlarged view of the distal end cap member as seen from the proximal side, and FIG. 10 shows a view of the plunger rod and of the guide tube as seen from the proximal side.

The drive unit in the depicted embodiment comprises the plunger rod 41 engaging, during medicament delivery, into the medicament container from distally, and further comprises a guide tube 53.

The base unit comprises, in addition to the housing sleeve 2, the distal end cap member 5 and the container housing 6 (these parts together constituting the housing) also a revolving member 42. The revolving member has a distal head 43 and a shaft 44 extending towards proximally, through an end plate hole 61 of the housing sleeve's 2 distal end plate 16, from the distal head 43. Helical grooves 45 extend along the shaft 44.

The distal end cap member 5 has, towards proximally, a shallow indentation that defines an inner control surface 72 with a plurality of teeth 73. In the assembled state, the distal head 43 of the revolving member is placed in the shallow indentation. The relative dimensions of the revolving member 42 and its striker 71 on the one hand and of the control surface 72 on the other hand are chosen in a manner that the striker 71 abuts against the control surface 72 and is thereby deflected towards radially-inwardly so that its resilience presses it against the control surface 72.

As soon as the drive locking member 26 ceases to block the drive unit in its initial position relative to the housing, a drive spring 51 accommodated in an interior of the plunger rod 41 and proximally abutting against the distal end plate 16 of the housing sleeve 2 acts to move the plunger rod 41 towards proximally relative to the base unit. The guide tube 53 moves with the plunger rod 41, due to a flange-like extension 81 of the guide tube 53 cooperating with an indentation 82 in the proximal end face of the plunger rod 41 (see FIG. 10). The revolving member 42 is prevented from axially moving together with the drive unit because its distal head 43 abuts against the distal end plate 16 (more in particular: against an annular rib 62 extending from the distal end plate 16) of the housing. Thus, the axial movement of the drive unit will involve a relative movement between the guide tube 53 and the revolving member 42.

At the same time, the guide tube 53 cannot rotate around the axis, because the flange-like extension 81 fixes its orientation relative to the plunger rod 41, and the orientation of the plunger rod is fixed relative to the housing by axial grooves 83 into which guiding features of the housing (for example of the container housing) engage. Because of engaging protrusions 46 of the guide tube 53 engaging into the helical grooves 45 extending along the shaft 44 of the revolving member 42, the relative movement between the guide tube 53 and the revolving member 42 will thus cause a rotation around the longitudinal axis 20, in the direction shown by the arrow in FIG. 8.

The rotation of the revolving member 42 will cause the striker 71 to move along the control surface over the teeth. The teeth are shaped asymmetrically (see FIG. 9) so that the striker 71 first moves along a ramp 74 and is thereby further deflected radially inwardly and then, when reaching a radial face 75 of the tooth 73, may jump back due to its resilience, thereby strike against the control surface 72 and cause a ‘click’-like sound.

In the illustrated embodiment, the teeth are arranged at unequal distances. Especially, at the beginning of its path during the rotation of the revolving member 42, the striker has to move over longer distances from tooth to tooth (see the section of the control surface 72 depicted at the bottom in FIG. 9), whereas towards the end of its path the distances between the teeth diminish. This compensates the effect that the velocity of the drive unit diminishes during its axial movement into the proximal direction, because due to Hook's law the spring force of the drive spring diminishes as the spring extends.

Various other embodiments may be envisaged. For example, it is not necessary that the revolving member with its shaft 44 is an inner element encompassed by the guide tube 53 of the drive unit. Rather, as an alternative, the revolving member could have a tube-shaped portion encompassing a shaft of the drive unit. The helical grooves (or helical ridges) could then be arranged on an inner surface of the revolving member. Also, the positions of the helical features (grooves, ridges or similar) and of the engaging features could be exchanged, i.e. the drive unit (for example the guide tube or a shaft) could have the helical features. If the engaging features are themselves helical, the cooperation is thread like.

Further, while in the described embodiment the revolving member belongs to the base unit—having an axial position that is fixed relative to the housing and the structure that causes it to rotate belongs to the drive unit, it would in principle also possible to reverse this, i.e., the revolving member could move with the drive unit. In this case, the cooperating structure that cooperates with the revolving member to generate the signals would move with the base unit, too, and/or would have a sufficient axial extension.

Even more, while in the depicted embodiment, the revolving member has the striker and the cooperating structure (of the distal end cap member in the shown embodiment) has the control surface with the teeth, this readily be interchanged.

Also, the biasing member causing the drive force is not necessarily a (helical) drive spring but could for example also be a gas cartridge or any other energy storing element.

Even further, the plunger rod 41 and the guide tube 53 (or, in other embodiments, a shaft in the latter's place) do not need to be separate parts but could be one-piece with each other. The drive unit will then not be an assembly of different parts but consist of a single part only, —the plunger rod with a tube or shaft structure.

The same holds true for other parts described in the present document, which parts do not move relative to each other: also these may optionally be combined to be one-piece with each other.

The drug delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjügren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin generelated peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, Blymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies. Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumabpertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator drug adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g. diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, humanderived, or recombinant hyaluronidase enzyme), concentration modifiers or enhancers, stabilizers, buffers, or other excipients. Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, MiniCHOP, Maxi-CHOP, VR-CAP, Dose-Dense CHOP, EPOCH, Dose-Adjusted EPOCH, REPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DTPACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-14. (canceled)

15. A medicament delivery device for accommodating a medicament container, the medicament delivery device comprising a base unit with a housing extending along a longitudinal axis and a drive unit arranged to be subject to an expelling movement relative to the base unit into a proximal direction, to act on the medicament container for expelling a medicament therefrom, wherein one of the drive unit and of the base unit further comprises a revolving member, and the other one comprises a cooperating structure, wherein the revolving member is mounted to be caused to rotate upon the expelling movement of the drive unit, and wherein one of the revolving member and the cooperating structure comprises a striker, and the other one comprises a control surface with a plurality of teeth, wherein, when the revolving member rotates, the striker moving along the control surface and moving over the teeth causes audible signals.

16. The medicament delivery device according to claim 15, further comprising a helical feature and an engaging feature engaging with the helical feature, wherein one of the helical feature and of the engaging feature is a feature of the revolving member, and wherein the expelling movement includes a relative axial movement of the helical feature and the engaging feature, whereby the expelling movement causes the revolving member to rotate.

17. The medicament delivery device according to claim 16, wherein the helical feature is a helically running groove, and the engaging feature is an engaging protrusion engaging into the helically running groove, or the helical feature is a helically running ridge, and the engaging feature is an engaging notch into which the helically running ridge engages.

18. The medicament delivery device according to claim 16, wherein the helical feature extends along a shaft or tube of the revolving member.

19. The medicament delivery device according to claim 15, wherein both the revolving member and the cooperating structure belong to the base unit, whereby the revolving member rotates, during the expelling movement, relative to the housing but does not move axially.

20. The medicament delivery device according to claim 19, wherein the cooperating structure is stationary relative to the housing.

21. The medicament delivery device according to claim 20, wherein the striker is an outward extension of a distal head of the revolving member, the control surface is an inner surface of the cooperating structure, the striker is deflected towards radially inwardly by abutting against the control surface, and the revolving member further comprises the shaft extending towards proximally from the distal head.

22. The medicament delivery device according to claim 21, wherein the housing comprises a housing sleeve, wherein the housing sleeve has a distal end plate with an end plate hole, and wherein the distal head is arranged distally of the distal end plate, and the shaft extends through the end plate hole, whereby the distal end plate fixes an axial position of the revolving member and prevents it from moving towards proximally.

23. The medicament delivery device according to claim 15, wherein the striker is resilient and is deflected resiliently by the striker moving over the teeth when the revolving member rotates.

24. The medicament delivery device according to claim 15, wherein the teeth are arranged at unequal distances.

25. The medicament delivery device according to claim 15, wherein the teeth are asymmetrical with respect to circumferential directions, the teeth having a ramp and a radial face or undercut face, so that when the revolving member rotates, the striker is lifted by the ramp against an elastic force and is allowed to jump back when it reaches the radial face or undercut face, wherein the jumping back of the striker and hitting against the control surface causes a click sound as one of the signals.

26. The medicament delivery device according to claim 15, wherein the drive unit comprises a plunger rod equipped to act on a plunger or stopper of the medicament container to move towards proximally during the expelling movement to expel the medicament from the medicament container.

27. The medicament delivery device according to claim 15, further comprising a biasing member equipped to move the drive unit towards distally during the expelling movement.

28. A medicament delivery assembly, comprising the medicament delivery device according to claim 15, and further comprising the medicament container assembled with the medicament delivery device.
